(19) United States
(12) Patent Application Publication
(10) Pub. No.: US 2009/0176699 A1
(43) Pub. Date: Jul. 9, 2009

(54) Inhibitors Based on Fusion, Hr1 and Hr2 Sequences in Bacterial Adhesin

(75) Inventors: Vega Masignani, Siena (IT); Rino Rappuoli, Castelnuoevo Berardenga (Siena) (IT); Barbara Capecchi, Pisa (IT); Silvana Savino, Florence (IT); Maria Beatrice Arico, Pogfibonsi (Siena) (IT)

Correspondence Address: NOVARTIS VACCINES AND DIAGNOSTICS INC., INTELLECTUAL PROPERTY R338, P.O. BOX 8097, Emeryville, CA, 94662-8097, US

(73) Assignee: CHIRON SRL, Siena (IT)

(21) Appl. No.: 11/631,807

(22) PCT Filed: Jul. 6, 2005
(86) PCT No.: PCT/IB05/02320

§ 371 Date: Jun. 27, 2007

(30) Foreign Application Priority Data
Jul. 6, 2004 (GB) 0415160.1

(51) Int. Cl. A61K 38/16; C07K 14/22; C12N 15/31; C12N 1/21

(52) U.S. Cl. 514/12; 530/324; 536/23.7; 435/252.3

(57) ABSTRACT

A known surface adhesin (NadA) in Neisseria meningitidis contains sequences which correspond to the fusion peptide, HR1 repeat and HR2 repeat seen in the envelope protein of viruses. Fusion inhibitors may thus be used to inhibit meningococcal infection, and the invention provides a compound that can bind to the heptad repeat sequence(s) HR1 and/or HR2 of the NadA adhesin on the surface of a meningococcus, thereby inhibiting the ability of the meningococcus either to infect a host organism or to spread an existing infection.

### TECHNICAL FIELD

This invention is in the field of antibacterials, particularly for preventing meningococcal infection.

### BACKGROUND ART

Neisseria meningitidis is a Gram-negative encapsulated bacterium which colonises the upper respiratory tract of approximately 10% of human population. Approximately once in every 10,000 colonised people (or once in 100,000 population) the bacterium enters the blood stream where it multiplies and causes sepsis. From the blood stream the bacterium can cross the blood-brain barrier and cause meningitis. Both diseases are devastating and can kill 5-15% of affected children and young adults within hours, despite the availability of effective antibiotics. Up to 25% of those who survive are left with permanent sequelae.

There has been widespread work on producing vaccines for preventing meningococcal infection {1}, but very little work on providing non-immunologically-based inhibitors of infection. It is an object of the invention to provide such non-immunologically-based inhibitors of meningococcal infection.

### DISCLOSURE OF THE INVENTION

The entry of enveloped viruses into target host cells requires their respective lipid bilayer membranes to fuse. The mechanism of HIV entry has been described in detail: binding of the HIV envelope glycoprotein gp120 to the CD4+ receptor on human target cells induces conformational changes that enable gp120 to interact with a chemokine receptor on the host cell; binding of gp120 to the coreceptor causes subsequent conformational changes in the viral transmembrane glycoprotein gp41, exposing the “fusion peptide” of gp41, which inserts into the cell membrane; a helical region of gp41, called HR1, then interacts with a similar helical region, HR2, on gp41, resulting in a “zipping” together of the two helices and mediating the fusion of cellular and viral membranes.

Enfuvirtide (also known as “T-20” or “Fuzeon™” {2}) is the prototypic “fusion inhibitor” anti-HIV drug. It is a linear 36-amino acid synthetic peptide that inhibits the HIV/T-cell interaction by binding to the HR1 heptad-repeat region in gp41 and preventing the conformational changes required for membrane fusion. Enfuvirtide is based on the HR2 sequence and is believed to act as a competitive inhibitor of the natural HR1/HR2 interaction.

This membrane fusion mechanism is typical for viruses, but the inventors have found that a known surface adhesin (NadA) in the Neisseria meningitidis bacterium {3} contains sequences which correspond to the fusion peptide, the HR1 repeat and the HR2 repeat (see FIG. 1). The sequences within NadA do not have any significant sequence similarity to the viral sequences, but were instead identified based on structural similarity of NadA to the SARS coronavirus spike protein. The surprising finding suggests that fusion inhibitors could be used to inhibit meningococcal infection.

### Binders of NadA HR1 and HR2

Thus the invention provides a compound that can bind to the heptad repeat sequence(s) HR1 and/or HR2 of the NadA adhesin on the surface of a meningococcus, thereby inhibiting the ability of the meningococcus either to infect a host organism or to spread an existing infection.

The HR1 region within NadA is, using as a reference the MC58 strain sequence (SEQ ID NO: 1), located between residues 117-152. The HR2 region, again with reference to strain MC58, is located between residues 261-299. The corresponding coordinates in other strains can be identified by simple alignments with the MC58 sequence.

Thus the invention also provides a compound that can bind to the HR1 and/or HR2 region(s) of the NadA adhesin on the surface of a meningococcus, wherein said HR1 sequence is, numbered according to the NadA sequence in strain MC58, located between residues 117-152, and wherein said HR2 sequence is, numbered according to the NadA sequence in strain MC58, located between residues 261-299. Strain MC58 is the strain which was used for sequencing the serogroup B genome {4} and it is widely available (e.g. ATCC BAA-335).

Haemophilus influenzae Biogroup aegyptius

As well as identifying HR1 and HR2 sequences in the NadA adhesin from N. meningitidis, the inventors have found HR1 and HR2 sequences in the HadA adhesin {5; SEQ ID NO: 35 herein} from H. influenzae biogroup aegyptius, the causative agent of Brazilian purpuric fever (BPF). The sequences within HadA do not have any significant sequence similarity to the viral sequences, but were instead identified based on structural similarity to NadA. The surprising similarity to viral HR sequences suggests that fusion inhibitors could be used to inhibit infection by H. influenzae.

Thus the invention provides a compound that can bind to the heptad repeat sequence(s) HR1 and/or HR2 of the HadA adhesin on the surface of a haemophilus bacterium (particularly H. influenzae, and more particularly boigroup aegyptius), thereby inhibiting the ability of the haemophilus either to infect a host organism or to spread an existing infection.

The HR1 region within HadA is, using as a reference the F3031 strain sequence (SEQ ID NO: 35), located between residues 71-91. The HR2 region, again with reference to strain F3031, is located between residues 120-183. The corresponding coordinates in other strains can be identified by simple alignments with the F3031 sequence.

Thus the invention also provides a compound that can bind to the HR1 and/or MR2 region(s) of the HadA adhesin on the surface of a haemophilus, wherein said HR1 sequence is, numbered according to the HadA sequence in strain F3031, located between residues 71-91, and wherein said HR2 sequence is, numbered according to the HadA sequence in strain F3031, located between residues 120-183. Strain F3031 is a BPF clone {6} and it is widely available (e.g. ATCC 49252).

### Oligopeptides

The compounds of the invention will typically be oligopeptides e.g. a peptide consisting of no more than z amino acids, where z is 50 or less (e.g. 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, etc.).

The invention provides an oligopeptide comprising a fragment of an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 39 and SEQ ID NO: 40, where the fragments consists of n consecutive amino acids from said SEQ ID, and where n is 5 or more (e.g. 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, etc.).

The invention also provides an oligopeptide comprising a fragment of an amino acid sequence selected from the group consisting of: SEQ ID NO: 12, SEQ ID NO: 13, SEQ ID NO: 14, SEQ ID NO: 15, SEQ ID NO: 16, SEQ ID NO: 17, SEQ ID NO: 39 and SEQ ID NO: 40, where the fragments consists of n consecutive amino acids from said SEQ ID, and where n is 5 or more (e.g. 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, etc.), provided that said fragment includes m amino acid substitutions when compared to said SEQ ID, where m is an integer between 1 and n/4.

The value of m is preferably 1, 2, 3, 4, 5, 6, 7, 8, 9 or 10. The m amino acids are typically substituted by A, C, D, E, F, G, H, I, K, L, M, N, P, Q, R, S, T, V, W, or Y. Each of the m substitutions may be the same or different as the others. The substitution is preferably by G or, more preferably, by A. The substituting amino acid may be an L- or a D- amino acid but, where the other n-m amino acids all share a single stereo-configuration (i.e. all D- or all L-), the substituting amino acid preferably also has that stereo-configuration (although, of course, G has no stereoisomers).

Where the fragment of n amino acids includes a C, the value of m is preferably at least 1 such that the C is substituted for another amino acid, such as S. Removal of C in this way can improve resistance to oxidation.

Preferred fragments of SEQ ID NO: 12 are also fragments of SEQ ID NO: 5. Preferred fragments of SEQ ID NO: 13 are also fragments of SEQ ID NO: 6. Preferred fragments of SEQ ID NO: 14 are also fragments of SEQ ID NO: 7. Preferred fragments of SEQ ID NO: 15 are also fragments of SEQ ID NO: 8. Preferred fragments of SEQ ID NO: 16 are also fragments of SEQ ID NO: 10. Preferred fragments of SEQ ID NO: 17 are also fragments of SEQ ID NO: 11. Preferred fragments of SEQ ID NO: 39 are also fragments of SEQ ID NO: 37. Preferred fragments of SEQ ID NO: 40 are also fragments of SEQ ID NO: 38.

Particularly preferred oligopeptides comprise or consist of one of the following amino acid sequences: SEQ ID NOs: 4, 5, 6, 7, 8, 9, 10, 11, 29, 30, 31, 37 and 38.

### Polypeptides

The compound of the invention can be an polypeptide e.g. consisting of between 2 and 1000 amino acids. The polypeptide preferably consists of no more than 250 amino acids (e.g. no more than 225, 200, 190, 180, 170, 160, 150, 140, 130, 120, 110, 100, 95, 90, 80, 70, 60, or no more than 50).

The polypeptide may have the formula NH₂-A-(B—C)ₙ-D-COOH, wherein: n is an integer between 1 and 5, -A- is an optional N-terminus sequence consisting of a amino acids; (each) -B- is an amino acid sequence comprising a fragment of b consecutive amino acids from SEQ ID NO: 12, SEQ ID NO: 13, SEQ ID NO: 14, SEQ ID NO: 15, SEQ ID NO: 16, SEQ ID NO: 17, SEQ ID NO: 39 and/or SEQ ID NO: 40; (each) -C- is an optional linker sequence consisting of c amino acids; and -D- is an optional C-terminus sequence consisting of d amino acids. The value of b is 5 or more (e.g. 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, etc.). Preferred fragments are as defined above (i.e. SEQ ID NOs: 4-11 and 29-31).

In some polypeptides, the amino acid sequence of the (or of one or more of each) -B- moiety may contain m amino acid substitutions, where m is an integer between 1 and n/4, as defined above.

Each of the n instances of -B- can be the same as or different from another -B-.

The value of a is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100; 150, 200, 250, 300, 350, 400, 450, 500, etc.), but can be zero (i.e. -A- is absent). Examples of typical -A- moieties include leader sequences to direct protein trafficking, or short peptide sequences which facilitate cloning or purification (e.g. histidine tags i.e. Hisₙ where n=3, 4, 5, 6, 7, 8, 9, 10 or more). Other suitable N-terminal amino acid sequences will be apparent to those skilled in the art.

The value of d is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. -D- is absent). Examples of typical -D- moieties include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminal amino acid sequences will be apparent to those skilled in the art.

The value of a+d may be 0 or greater (e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). It is preferred that the value of a+d is at most 1000 (e.g. at most 900, 800, 700, 600, 500, 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8, 7, 6, 5, 4, 3, 2).

The amino acid sequence of -A- typically shares less than x % sequence identity to the a amino acids which are N-terminal of sequence -B- in a NadA (or, where applicable, HadA) sequence (e.g. in SEQ ID NO: 1 or 2 or 35), and the amino acid sequence of -D- typically shares less than y % sequence identity to the d amino acids which are C-terminal of sequence -B- in a NadA (or HadA) sequence (e.g. in SEQ ID NO: 1 or 2 or 35). In general, the values of x and y are both 60 or less (e.g. 50, 40, 30, 20, 10 or less). The values of x and y may be the same as or different from each other.

The value of each c is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. -C- is absent). The values of each n instances of c may be the same as or different from each other.

Each of the n instances of C can be the same as or different from another C.

The amino acid sequence of -Cₙ- (i.e. the n^{th} instance of moiety -C-) typically shares less than z % sequence identity to the c amino acids which are C-terminal of sequence -Bₙ- in a NadA (or HadA) sequence (e.g. in SEQ ID NO: 1 or 2 or 35). In general, the value of z is 60 or less (e.g. 50, 40, 30, 20, 10 or less). Where n>1, the values of each z may be the same as or different from each other.

The value of n is preferably 1, such that the polypeptide has formula NH₂-A-B—C-D-COOH.

### Peptides of the Invention

Polypeptides of the invention (including oligopeptides, collectively “peptides”) may be linear, branched or cyclic, but they are preferably linear chains of amino acids. Where cysteine residues are present, peptides of the invention may be linked to other peptides via disulfide bridges. Peptides of the invention may comprise L-amino acids and/or D-amino acids. The inclusion of D-amino acids may be preferred in order to confer resistance to mammalian proteases.

The N-terminus residue of a peptide of the invention may be covalently modified. Suitable covalent groups include, but are not limited to: acetyl (as in Fuzeon™); a hydrophobic group; carbobenzoxyl; dansyl; T-butyloxycarbonyl; amido; 9-fluorenylmethoxy-carbonyl (FMOC); a lipid; a fatty acid; polyethylene; carbohydrate; etc.

Similarly, the C-terminus residue of a peptide may be covalently modified (e.g. carboxamide, as in Fuzeon™, etc.). Suitable covalent groups include, but are not limited to: acetyl (as in Fuzeon™); a hydrophobic group; amido; carbobenzoxyl; dansyl; T-butyloxycarbonyl; 9-fluorenylmethoxy-carbonyl (FMOC); a lipid; a fatty acid; polyethylene; carbohydrate; etc.

Peptides of the invention may be produced by various means.

A preferred method for production involves in vitro chemical synthesis {7,8}. Solid-phase peptide synthesis is particularly preferred, such as methods based on t-Boc or Fmoc {9} chemistry. Enzymatic synthesis {10} may also be used in part or in full.

As an alternative to chemical synthesis, biological synthesis may be used e.g. the peptides may be produced by translation. This may be carried out in vitro or in vivo. Biological methods are in general restricted to the production of peptides based on L-amino acids, but manipulation of translation machinery (e.g. of aminoacyl-tRNA molecules) can be used to allow the introduction of D-amino acids (or of other non-natural amino acids, such as iodotyrosine or methylphenylalanine, azidohomoalanine, etc.) {11}. Where D-amino acids are included in peptides of the invention, however, it is preferred to use chemical synthesis.

Production of peptides by biological means gives peptides with a N-terminus methionine residue. Where the N-terminus of a peptide of the invention is not a methionine then this residue (and any other extraneous residues) will have to be removed e.g. by proteolytic digestion.

To facilitate biological synthesis of peptides, the invention provides nucleic acid that encodes a peptide of the invention. The nucleic acid may be DNA or RNA (or hybrids thereof), or their analogues, such as those containing modified backbones (e.g. phosphorothioates) or peptide nucleic acids (PNA). It may be single-stranded (e.g. mRNA) or double-stranded, and the invention includes both individual strands of a double-stranded nucleic acid (e.g. for antisense, priming or probing purposes). It may be linear or circular. It may be labelled. It may be attached to a solid support.

Nucleic acid according to the invention can, of course, be prepared in many ways e.g. by chemical synthesis (e.g. phosphoramidite synthesis of DNA) in whole or in part, by nuclease digestion of longer molecules, by ligation of shorter molecules, from genomic or cDNA libraries, by use of polymerases etc.

The invention provides vectors (e.g. plasmids) comprising nucleic acid of the invention (e.g. expression vectors and cloning vectors) and host cells (prokaryotic or eukaryotic) transformed with such vectors.

### Drug Design and Peptidomimetics

Peptides of the invention are useful antibacterials in their own right. However, they may be refined to improve anti-bacterial activity or to improve pharmacologically important features such as bio-availability, toxicology, metabolism, pharmacokinetics, etc. The peptides may therefore be used as lead compounds for further research and refinement.

Peptides of the invention can be used for designing peptidomimetic molecules {e.g. refs. 12 to 18} with anti-meningococcal or anti-haemophilus activity. These will typically be isosteric with respect to the peptides of the invention but will lack one or more of their peptide bonds. For example, the peptide backbone may be replaced by a non-peptide backbone while retaining important amino acid side chains.

The peptidomimetic molecule may comprise sugar amino acids {19}. Peptoids may be used.

To assist in the design of peptidomimetic molecules, a pharmacophore (i.e. a collection of chemical features and 3D constraints that expresses specific characteristics responsible for activity) can be defined for the KM peptides. The pharmacophore preferably includes surface-accessible features, more preferably including hydrogen bond donors and acceptors, charged/ionisable groups, and/or hydrophobic patches. These may be weighted depending on their relative importance in conferring activity {20}.

Pharmacophores can be determined using software such as CATALYST (including HypoGen or HipHop) {21}, CERIUS², or constructed by hand from a known conformation of a polypeptide of the invention. The pharmacophore can be used to screen structural libraries, using a program such as CATALYST. The CLIX program {22} can also be used, which searches for orientations of candidate molecules in structural databases that yield maximum spatial coincidence with chemical groups which interact with the receptor.

The binding surface or pharmacophore can be used to map favourable interaction positions for functional groups (e.g. protons, hydroxyl groups, amine groups, hydrophobic groups) or small molecule fragments. Compounds can then be designed de novo in which the relevant functional groups are located in substantially the same spatial relationship as in polypeptides of the invention.

Functional groups can be linked in a single compound using either bridging fragments with the correct size and geometry or frameworks which can support the functional groups at favourable orientations, thereby providing a peptidomimetic compound according to the invention. Whilst linking of functional groups in this way can be done manually, perhaps with the help of software such as QUANTA or SYBYL, automated or semi-automated de novo design approaches are also available, such as:
MCSS/HOOK {23, 24, 21}, which links multiple functional groups with molecular templates taken from a database.
LUDI {25, 21}, which computes the points of interaction that would ideally be fulfilled by a ligand, places fragments in the binding site based on their ability to interact with the receptor, and then connects them to produce a ligand.
MCDLNG {26}, which fills a receptor binding site with a close-packed array of generic atoms and uses a Monte Carlo procedure to randomly vary atom types, positions, bonding arrangements and other properties.
GROW {27}, which starts with an initial ‘seed’ fragment (placed manually or automatically) and grows the ligand outwards.
SPROUT {28}, suite which includes modules to: identify favourable hydrogen bonding and hydrophobic regions within a binding pocket (HIPPO module); select functional groups and position them at target sites to form starting fragments for structure generation (EleFAnT); generate skeletons that satisfy the steric constraints of the binding pocket by growing spacer fragments onto the start fragments and then connecting the resulting part skeletons (SPIDeR); substitute hetero atoms into the skeletons to generate molecules with the electrostatic properties that are complementary to those of the receptor site (MARABOU). The solutions can be clustered and scored using the ALLigaTOR module.
CAVEAT {29}, which designs linking units to constrain acyclic molecules.
LEAPFROG {30}, which evaluates ligands by making small stepwise structural changes and rapidly evaluating the binding energy of the new compound. Changes are kept or discarded based on the altered binding energy, and structures evolve to increase the interaction energy with the receptor.
GROUPBUILD {31}, which uses a library of common organic templates and a complete empirical force field description of the non-bonding interactions between a ligand and receptor to construct ligands that have chemically reasonable structure and have steric and electrostatic properties complimentary to the receptor binding site.
RASSE {32}

These methods identify antibacterial compounds. These compounds may be designed de novo, may be known compounds, or may be based on known compounds. The compounds may be useful antibacterials themselves, or they may be prototypes which can be used for further pharmaceutical refinement (i.e. lead compounds) in order to improve binding affinity or other pharmacologically important features (e.g. bio-availability, toxicology, metabolism, pharmacokinetics etc.).

The invention thus provides: (i) a compound identified using these drug design methods; (ii) a compound identified using these drug design methods, for use as a pharmaceutical; (iii) the use of a compound identified using these drug design methods in the manufacture of an antibacterial e.g. for preventing meningococcal or haemophilus infection; (iv) a method of treating a patient, comprising administering an effective amount of a compound identified using these drug design methods.

As well as being useful compounds individually, ligands identified in silico by the structure-based design techniques can also be used to suggest libraries of compounds for ‘traditional’ in vitro or in vivo screening methods. Important pharmaceutical motifs in the ligands can be identified and mimicked in compound libraries (e.g. combinatorial libraries) for screening for microbicidal and/or antiviral activity.

Attenuated Meningococci

The NadA adhesin forms surface-exposed oligomers on meningococcus and is involved adhesion to epithelial cells {41}. Adhesion is part of the pathogenic cycle in meningococcus, and its inhibition could attenuate the bacterium such that it cannot invade cells and cause disease, without loss of the bacterium's overall immunogenicity. One way of inhibiting adhesion according to the invention is to remove one or more of the HR1, HR2 or fusion peptide sequences from NadA.

Thus the invention provides a mutant NadA protein, wherein the mutant protein lacks one or more of the HR1, HR2 or fusion sequences.

The invention also provides a mutant NadA protein, wherein the mutant does not contain one or more of the following amino acid sequences: (i) a sequence which has at least p % identity to SEQ ID NO: 3; (ii) a sequence which has at least q % identity to SEQ ID NO: 5; (iii) a sequence which has at least r % identity to SEQ ID NO: 7; (iv) a sequence which has at least s % identity to SEQ ID NO: 10.

The value of p is 50 or more. The value of q is 50 or more. The value of r is 50 or more. The value of s is 50 or more. The values of p, q, r and s are independent of each other, and typical values are 60, 70, 80, 90, 95, 96, 97, 98, 99 or 100.

The amino acid sequences (i), (ii), (iii) and (iv) are preferably at least 10 amino acids long, and are more preferably at least 15 amino acids long.

The invention also provides a mutant NadA protein, comprising amino acid sequence -A-B-C-D-E-F-G-H-I-, wherein: -A- is an amino acid sequence with at least a % sequence identity to amino acids 26-116 of SEQ ID NO: 1; -B- is an amino acid sequence with at least b % sequence identity to amino acids 117-152 of SEQ ID NO: 1; -C- is an amino acid sequence with at least c % sequence identity to amino acids 153-180 of SEQ ID NO: 1; -D- is an amino acid sequence with at least d % sequence identity to amino acids 181-199 of SEQ ID NO: 1; -E- is an amino acid sequence with at least e % sequence identity to amino acids 200-260 of SEQ ID NO: 1; -F- is an amino acid: sequence with at least f % sequence identity to amino acids 261-275 of SEQ ID NO: 1; -G- is an amino acid sequence with at least g % sequence identity to amino acids 276-277 of SEQ ID NO: 1; -H- is an amino acid sequence with at least h % sequence identity to amino acids 278-299 of SEQ ID NO: 1; -I- is an amino acid sequence with at least i % sequence identity to amino acids 300-364 of SEQ ID NO: 1, provided that at least one of -B-, -D-, -F- or -H- is not present in said protein.

The value of a is 50 or more. The value of b is 50 or more. The value of c is 50 or more. The value of d is 50 or more. The value of e is 50 or more. The value off is 50 or more. The value of g is 50 or more. The value of h is 50 or more. The value of i is 50 or more. The values of a, b, c, d, e, f g, h and i are independent of each other, and typical values are 60, 70, 80, 90, 95, 96, 97, 98, 99 or 100.

The invention also provides nucleic acid encoding these mutant NadA proteins. The invention also provides a meningococcus which expresses said nucleic acid (a), which displays said mutant NadA protein on its surface, and which cannot bind and/or enter human epithelial cells.

The mutations can be introduced into target meningococci by homologous recombination (e.g. using the isogenic deletion technique) to remove the native nadA sequence.

### Attenuated Haemophilus

One way of inhibiting haemophilus adhesion according to the invention is to remove one or more of the HR1, HR2 or fusion peptide sequences from HadA. Thus the invention provides a mutant HadA protein, wherein the mutant protein lacks one or more of the HR1, HR2 or fusion sequences.

The invention also provides a mutant HadA protein, wherein the mutant does not contain an amino acid sequence which has at least p % identity to SEQ ID NO: 35, where the value of p is 50 or more (e.g. 60, 70, 80, 90, 95, 96, 97, 98, 99 or 100). The amino acid sequence is preferably at least 10 amino acids long, and are more preferably at least 15 amino acids long.

The invention also provides a mutant HadA protein, comprising amino acid sequence -A-B-C-D-E-F-G-, wherein: -A- is an amino acid sequence with at least a % sequence identity to amino acids 27-50 of SEQ ID NO: 35; -B- is an amino acid sequence with at least b % sequence identity to amino acids 5167 of SEQ ID NO: 35; -C- is an amino acid sequence with at least c % sequence identity to amino acids 68-70 of SEQ ID NO: 35; -D- is an amino acid sequence with at least d % sequence identity to amino acids 71-91 of SEQ ID NO: 35; -E- is an amino acid sequence with at least e % sequence identity to amino acids 92-119 of SEQ ID NO: 35; -F- is an amino acid sequence with at least f % sequence identity to amino acids 120-183 of SEQ ID NO: 35; -G- is an amino acid sequence with at least g % sequence identity to amino acids 184-256 of SEQ ID NO: 35, provided that at least one of -B-, -D- or -F- is not present in said protein.

The value of a is 50 or more. The value of b is 50 or more. The value of c is 50 or more. The value of d is 50 or more. The value of e is 50 or more. The value off is 50 or more. The value of g is 50 or more. The values of a, b, c, d, e, f and g are independent of each other, and typical values are 60, 70, 80, 90, 95, 96, 97, 98, 99 or 100.

The invention also provides nucleic acid encoding these mutant HadA proteins. The invention also provides a haemophilus which expresses said nucleic acid (a), which displays said mutant HadA protein on its surface, and which cannot bind and/or enter human epithelial cells.

The mutations can be introduced into target haemophilus by homologous recombination (e.g. using the isogenic deletion technique) to remove the native hadA sequence.

### Pharmaceutical Compositions

The invention provides a pharmaceutical composition comprising (a) a peptide of the invention and (b) a pharmaceutical carrier.

Component (a) is the active ingredient in the composition, and this is present at a therapeutically effective amount e.g. an amount sufficient to inhibit meningococcal or haemophilus infection. The precise effective amount for a given patient will depend upon their size and health, the nature and extent of infection, and the composition or combination of compositions selected for administration. The effective amount can be determined by routine experimentation and is within the judgment of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg. Pharmaceutical compositions based on peptides are well known in the art (e.g. FUZEON™). Peptides may be included in the composition in the form of salts and/or esters.

Carrier (b) can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles. Liposomes are suitable carriers. A thorough discussion of pharmaceutical carriers is available in ref. 33.

Meningococcal and haemophilus infections affect various areas of the body and so the compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops, as a spray, or as a powder {e.g. 34}. The composition may be included in a mouthwash. The composition may be lyophilised.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe or an inhaler.

Peptides of the invention may be co-administered with one or more antibiotics, preferably those which are active against meningococcus and/or haemophilus. Compositions of the invention may thus include one or more antibiotics.

### Medical Treatments and Uses

The invention provides a compound of the invention for use as a medicament. The invention also provides a method for treating a patient suffering from a meningococcal and/or haemophilus infection, comprising administering to the patient a pharmaceutical composition of the invention. The invention also provides the use of a compound of the invention in the manufacture of a medicament for treating a patient.

The patient is preferably a human. The human may be an adult or, preferably, a child. A composition intended for children may also be administered to adults e.g. to assess safety, dosage, immunogenicity, etc.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermal {e.g. see ref. 35} or transcutaneous {e.g. see refs. 36 & 37}, intranasal {e.g. see ref. 38}, ocular, aural, pulmonary or other mucosal administration.

Dosage treatment can be a single dose schedule or a multiple dose schedule.

The uses and methods of the invention can be used therapeutically (e.g. for treating an existing bacterial and meningococcal meningitis, or BPF) or prophylactically (e.g. in a situation where contact with microbes is expected and where establishment of infection is to be prevented). Therapeutic use is preferred, and efficacy of treatment can be tested by monitoring bacterial titres after administration of the pharmaceutical composition of the invention, or by monitoring symptoms.

### Processes

The invention also provides a process for producing a peptide of the invention, comprising the step of culturing a host cell transformed with nucleic acid of the invention under conditions which induce expression of the peptide.

The invention provides a process for producing a peptide of the invention, comprising the step of synthesising the peptide by chemical means. The peptide may be synthesised in whole or in part by such chemical means.

### General

The term “comprising” encompasses “including” as well as “consisting of” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of reference 39. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in reference 40.

The use of “NH₂” and “COOH” in peptide sequences implies only the direction of the peptide chain from N-terminus to C-terminus, and does not imply that the N-terminus residue must have a free —NH₂ group or that the C-terminus must have a free —COOH group (although nor is such a situation excluded). On the contrary, the N- and C-termini may be covalently modified.

Compounds of the invention can preferably inhibit either (a) the interaction of NadA HR1 with NadA HR2, or (b) the interaction of HadA HR1 with HadA HR2.

The binding interaction between a compound of the invention and NadA/HadA is specific. Specificity in this context does not mean that the compound binds nothing other than NadA/HadA (e.g. it may bind other adhesins or surface proteins), but means that the compound binds to NadA/HadA above background (i.e. non-specific) levels. For instance, the compound binds to NadA/HadA more tightly than it binds to proteins such as albumins, globulins, etc.

### BRIEF DESCRIPTION OF DRAWINGS

FIG. 1 shows NadA from strain MC58, with regions of interest highlighted.

FIG. 2 shows a helical wheel analysis of SARS E2 (FIG. 2A) and NadA (FIG. 2B).

FIG. 3 shows models of conformational changes in (3A) influenza HA and (3B) HadA.

### BRIEF DESCRIPTION OF SEQUENCE LISTING

| SEQ ID NO: | Description |
|---|---|
| 1 | NadA from N. meningitidis strain MC58 |
| | (GenBank accession: AAF42321) |
| 2 | NadA from N. meningitidis strain 2996 |
| 3 | Fusion peptide from SEQ ID NO^{S}: 1 & 2 |
| 4 | HR1 (X at aa 24 is E or A) from SEQ ID NO^{S}: 1 & 2 |
| 5 | HR1 from SEQ ID NO: 1 |
| 6 | HR1 from SEQ ID NO: 2 |
| 7 | HR2a from SEQ ID NO: 1 |
| 8 | HR2a from SEQ ID NO: 2 |
| 9 | HR2b (X: aa 4 is T or N; aa 6 is S or A; aa 7 is D or N) |
| | from SEQ ID NO^{S}: 1 & 2 |
| 10 | HR2b from SEQ ID NO: 1 |
| 11 | HR2b from SEQ ID NO: 2 |
| 12 | SEQ ID 5, extended 5aa both ways |
| 13 | SEQ ID 6, extended 5aa both ways |
| 14 | SEQ ID 7, extended 5aa both ways |
| 15 | SEQ ID 8, extended 5aa both ways |
| 16 | SEQ ID 10, extended 5aa both ways |
| 17 | SEQ ID 11, extended 5aa both ways |
| 18 | SARS coronavirus, E2 protein |
| 19-27 | Spike protein fusion sequences |
| 28 | NadA fusion sequence |
| 29-31 | Synthesised HR1, HR2a and HR2b sequences for NadA |
| 32 | Synthesised fusion sequence for NadA |
| 33 | Combined fusion sequence for NadA |
| 34 | SARS sequence |
| 35 | HadA |
| 36 | HadA fusion sequence |
| 37 | HadA HR1 |
| 38 | HadA HR2 |
| 39-40 | Synthesised HR1 and HR2 sequences for HadA |

### MODES FOR CARRYING OUT THE INVENTION

### Meningococcal NadA

Reference 3 discloses details of the Neisserial Adhesin A, a surface protein of Neisseria meningitidis. NadA sequences are given from 26 different meningococcal strains, including strains from serogroups A, B and C. The sequences were divided into three different alleles.

NadA was not seen in the hypervirulent lineage III of N. meningitidis, in N. gonorrhoeae, N. lactamica or N. cinerea. NadA is also absent from the published sequence of serogroup A meningococcal strain Z7491. The different sequences have been deposited in GenBank, and can also be seen in SEQ ID NOs: 1 to 14 of reference 41.

Based on the published sequences and characterisation, the skilled person will be able to identify the NadA sequence (or its absence) for any given strain of meningococcus.

SEQ ID NO:1 herein is the NadA sequence from strain MC58, which has allele “1”. SEQ ID NO:2 is the NadA sequence from strain 2996, which has allele “3”. An alignment of these two sequences is given below:

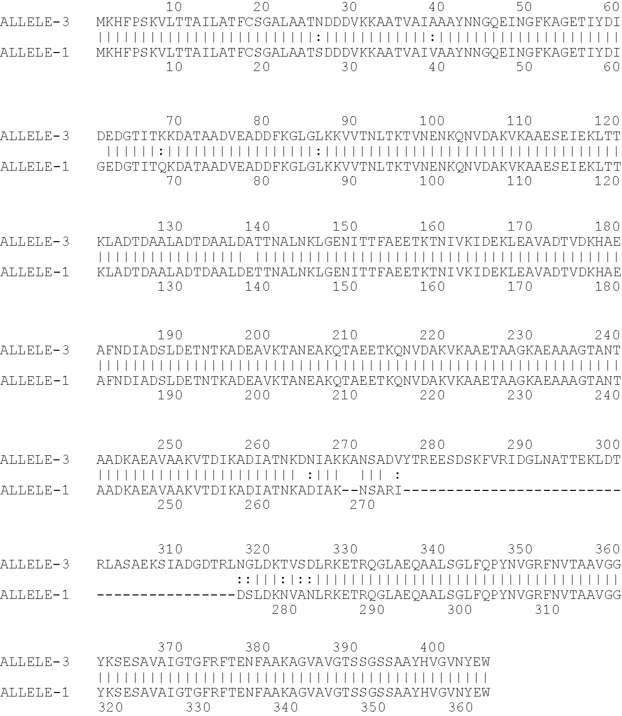

Reference 3 shows that NadA has a membrane anchor and that the protein assembles in the meningococcal membrane to form oligomers that associate via coiled-coil domains.

SARS Coronavirus spike Protein

The E2 spike protein of the SARS coronavirus has been reported. An amino acid sequence of this protein is given herein as SEQ ID NO:18. A CLUSTAL W alignment of SEQ ID NOS: 1 and 18, (i.e. NadA and E2) reveals less than 6% identity:

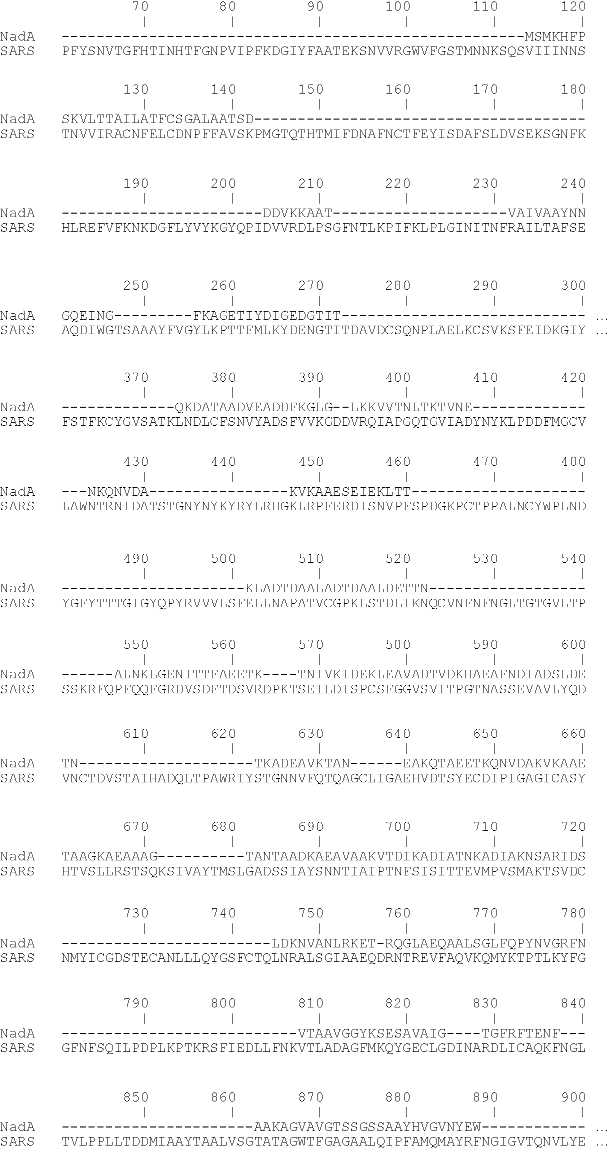

Thus, based purely on primary sequence, which is the usual criterion by which evolutionary relationships are judged, the 1255 mer viral SARS protein and the 364 mer bacterial NadA protein appear unrelated.

A secondary structure prediction for SARS E2 protein is given below, where C represents a coil, H represents a helix and E represents an extended sequence:

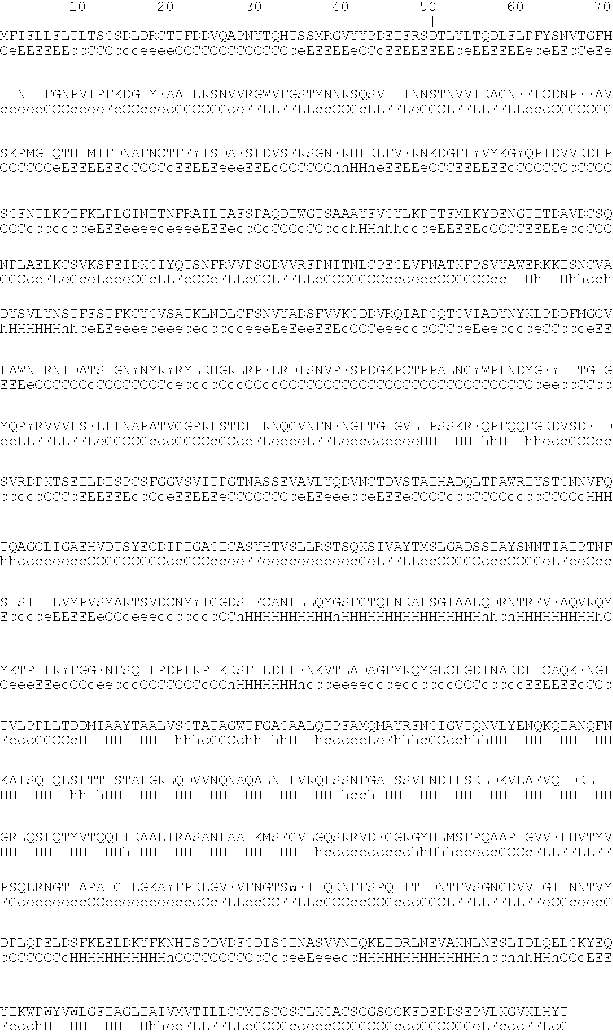

The secondary structure revealed for SARS E2 protein (and, indeed, for the fusion proteins of many other enveloped viruses) is similar to that seen in NadA:

MSMKHFPSKVLTTAILATFCSGALAATSDDDVKKAATVAIVAAYNNGQEINGFKAGETIYDIGEDGTITQ
CCCCCCCcHHHHHHHHHHHHhHhhhccCCHHHHHHHHHhhhhhhcCcceeecccCCeEeeccCCCCceec
KDATAADVEADDFKGLGLKKVVTNLTKTVNENKQNVDAKVKAAESEIEKLTTKLADTDAALADTDAALDE
chhhHHhhhHHhhhhCCCeeeehHHHHHHHhhhchHHHHHHHHHHHHHHHHHHHHHHhcccccchHhccc
TTNALNKLGENITTFAEETKTNIVKIDEKLEAVADTVDKHAEAFNDIADSLDETNTKADEAVKTANEAKQ
cHHHHHHhcCCHhHHHHhhccCccccchhHHHHHHHHHHHHHHHHHHHHHHHhhchHHHHHHHHhccHHH
TAEETKQNVDAKVKAAETAAGKAEAAAGTANTAADKAEAVAAKVTDIKADIATNKADIAKNSARIDSLDK
HHHHHHHHHHHHHHHHHHHHHHhhecCCCchHHHHhcccceEEEEehHHHHhcCCCccccCCcchHHHHH
NVANLRKETRQGLAEQAALSGLFQPYNVGRFNVTAAVGGYKSESAVAIGTGFRFTENFAAKAGVAVGTSS
HHHHHHHHHHHHHHHHHHHHHhcCCCCcceeEEEEEeCCCchhheeecCCccchhHHHHHhCCcEEEcCC
GSSAAYHVGVNYEW
CCcceeeeCeeecC

This similarity at the secondary structure level suggested to the inventors that NadA might share functional features with the viral spike protein. In particular, a HR2 sequence has recently been shown to inhibit coronavirus viral entry and membrane fusion {42}, as seen with FUZEON™ in HIV, and so the inventors looked at NadA to locate possible fusion, HR1 and HR2 sequences.

### NadA Fusion and Heptad Repeat Sequences

The fusion peptide sequences for various virus spike proteins are shown below, followed by a consensus sequence:

| | | |
|---|---|---|
| MHV spike | (971) KMIASAFNNALGAIQDGFD | SEQ ID NO: 19 |
| BCV spike | (1015) KLIANAFNNALDAIQEGFD | SEQ ID NO: 20 |
| FIPV spike | (1079) KILANAFNNAIGNITLALG | SEQ ID NO: 21 |
| TGEV spike | (1060) QILASAFNQAIGNITQSFG | SEQ ID NO: 22 |
| Avian IBV | (795) EKIAASFNKAIGHMQEGFR | SEQ ID NO: 23 |
| spike | | |
| HCoV 229E | (792) KILAASFNKAMTNIVDAFT | SEQ ID NO: 24 |
| spike | | |
| HCoV 0C43 | (1005) KLIANAFNNALYAIQEGFD | SEQ ID NO: 25 |
| spike | | |
| SARS chiron | (903) KQIANQFNKAISQIQESLT | SEQ ID NO: 26 |
| spike | | |
| Consensus | (1123) KIIANAFNNAIGNIQEGF | SEQ ID NO: 27 |

This consensus sequence was used to identify a fusion sequence in NadA (SEQ ID NOs: 1 & 2):

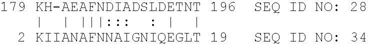

Taking into account the similarity to the SARS fusion sequence and amphipaticity of the helical sequence of a peptide sequence then sequences SEQ ID NO: 28 and SEQ ID NO: 3 can be identified as fusion sequences. In combination these two sequences give SEQ ID NO: 33.

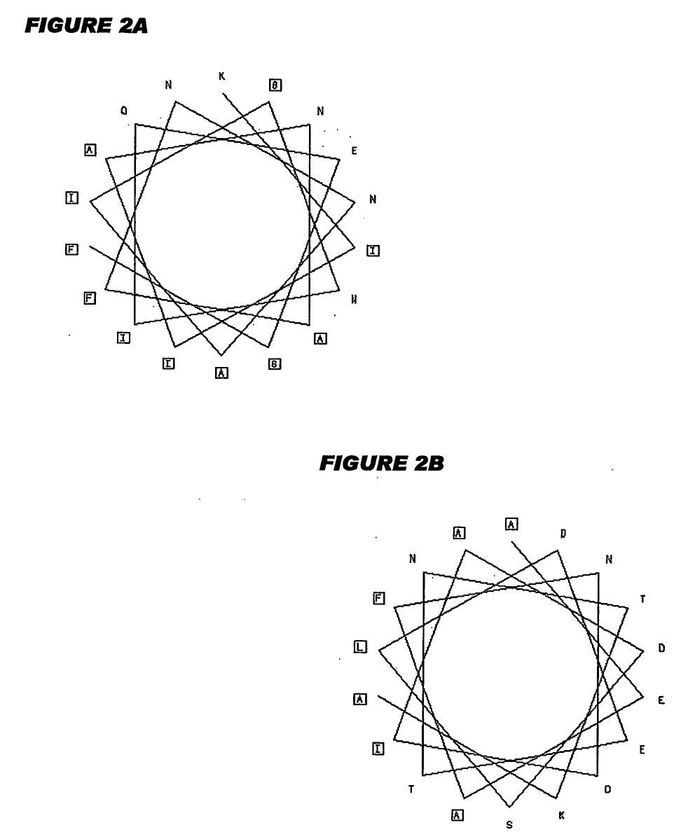

A helical wheel projection of E2 (residues 903-921) and NadA (residues 181-199) is shown in FIG. 2. Hydrophobic faces are clearly seen (boxed residues).

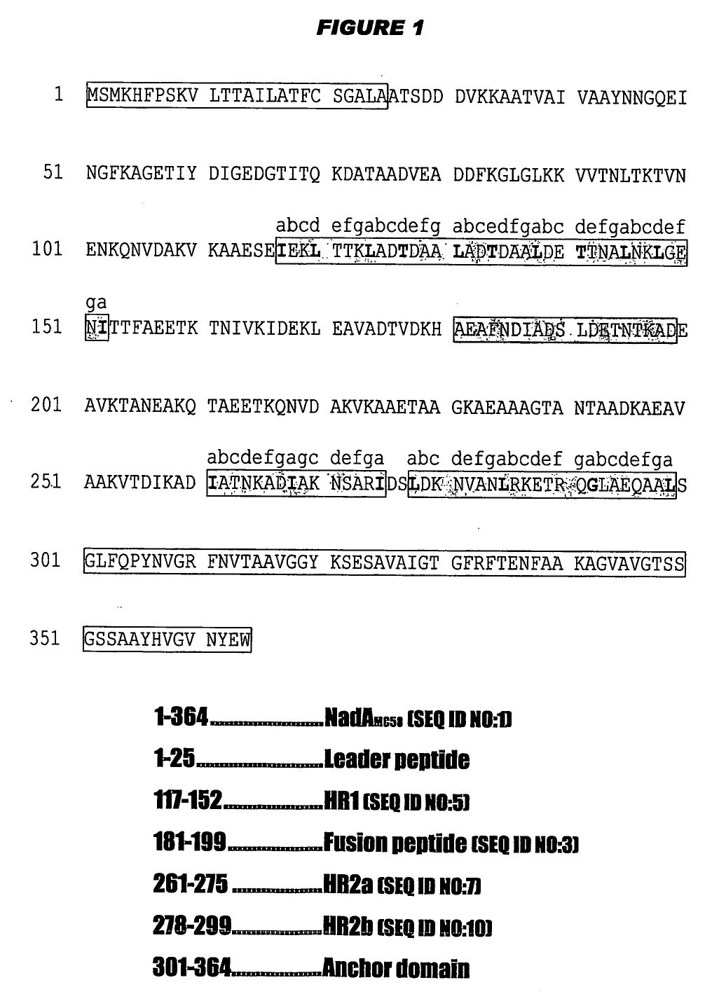

Heptad repeat sequences were identified in NadA (SEQ ID NO: 1) as shown in FIG. 1.

The HR1 sequence maps to residues 117-152 of SEQ ID NO: 1, showing a regular abcdefg heptad repeat with appropriate residues at positions a and d. The HR1 sequence in SEQ ID NO: 2 differs slightly by having an Ala/Glu substitution (compare SEQ ID NOs: 5 & 6; SEQ ID NO: 4).

Two possible HR2 sequences are seen, with the first (HR2a) being shorter than the second (HR2b). The HR2a sequence maps to residues 261-275 of SEQ ID NO: 1, and HR2b maps to residues 278-299. The HR2a sequence is in a region where the alignments of NadA alleles 1 and 3 show a clear gap, and this is reflected at the C-termini of HR2a sequences (compare SEQ ID NOs: 7 & 8). The HR2b sequences are downstream of the insertion and are more closely related (compare SEQ ID NOs: 10 & 11; SEQ ID NO: 9).

NadA HR1 and HR2 Peptides

Based on the surprising relationship between meningococcal NadA and the SARS coronavirus spike protein (see above), on the recently-identified efficacy of HR2 peptides in preventing coronavirus entry {42}, and on the known efficacy of HR2 peptides in preventing HIV activity (i.e. FUZEON™), the HR1 and HR2 sequences from NadA were chemically synthesised as oligopeptides for testing against meningococcus.

Sequences were taken from allele “3” of NadA (from SEQ ID NO:2). The HR1 oligopeptide is SEQ ID NO: 29. The HR2a oligopeptide is SEQ ID NO: 30. The HR2b oligopeptide is SEQ ID NO: 31. Each of these sequences is based on the “core” sequence (SEQ ID NOs: 6, 8 & 11) extended 3 amino acids in the N- and C-terminus directions.

An oligopeptide based on the fusion peptide was also prepared (SEQ ID NO: 32).

### HadA HR1 and HR2 Peptides

The full-length HadA sequence from BPF clone F3031 is given as SEQ ID NO: 35. Analysis of the sequence reveals a leader sequence (amino acids 1-26), a possible fusion sequence (51-67; SEQ ID NO: 36), a HR1 sequence (71-21; SEQ ID NO: 37), a HR2 sequence (120-183; SEQ ID NO: 38) and a membrane anchor (186-256). These features are indicated below, with underlining showing (i) hydrophobic residues in the fusion sequence or (ii) heptad repeat residues in HR1 and HR2:

| | |
|---|---|
| 1 | MKRNLLKQSVIAVLIGGTTVSNYALAQAQAQAQVKKDELSELKKQVKEMD |
| 51 | AAIDGILDDNIAYEAEVDAKLDQHSAALGRHTNRLNNLKTIAEKAKGDSS |
| 101 | EALDKIEALEEQNDEFLADITALEEGVDGLDDDITGIQDNISDIEDDINQ |
| 151 | NSADIATNTAAIATHTQRLDNLDNRVNNLNKDLKRGLAAQAALNGLFQPY |
| 201 | NVGKLNLTAAVGGYKSQTAVAVGTGYRYNENIAAKAGVAFTHGGSATYNV |
| 251 | GVNFEW* |

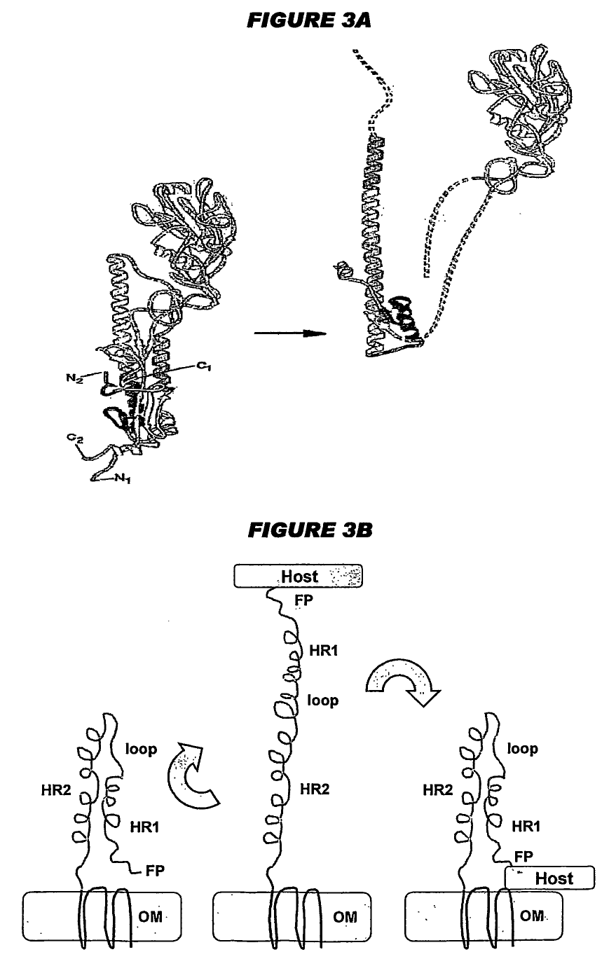

A model for the pH-dependent conformational change of influenza virus haemagglutinin is shown in FIG. 3A, and FIG. 3B shows a model showing how an equivalent conformational change in HadA (and by analogy NadA) could be involved in adhesion.

Synthetic sequences SEQ ID NO: 39 and SEQ ID NO: 40 were prepared for fusion studies.

It will be understood that the invention has been described by way of example only and modifications may be made whilst remaining within the scope and spirit of the invention.

### REFERENCES

(the contents of which are hereby incorporated by reference)
{1} Bethell & Pollard (2002) Expert Rev Vaccines 1:75-84.
{2} U.S. Pat. No. 5,464,933.
{3} Comanducci et al. (2002) J Exp Med 195:1445-1454
{4} Tettelin et al. (2000) Science 287:1809-1815.
{5} WO2004/113371.
{6} Smoot et al. (2002) Infect Immun 70:2694-99.
{7} Bodanszky (1993) Principles of peptide Synthesis (ISBN: 0387564314).
{8} Fields et al. (1997) Methods in Enzymology 289: Solid-Phase Peptide Synthesis. ISBN: 0121821900
{9} Chan & White (2000) Fmoc Solid Phase Peptide Synthesis ISBN: 0199637245.
{10} Kullmann (1987) Enzymatic Peptide Synthesis. ISBN: 0849368413.
{11} Ibba (1996) Biotechnol Genet Eng Rev 13:197-216.
{12} Kazmierski (1999) Peptidomimetics Protocols. ISBN: 0896035174.
{13} Kirshenbaum et al. (1999) Curr Opin Struct Biol 9:530-5.
{14} Abell (1999) Advances in Amino Acid Mimetics and Peptidomimetics. ISBN: 0762306149.
{15} U.S. Pat. No. 5,331,573 (Balaji).
{16} Goodman et al. (2001) Biopolymers 60:229-245.
{17} Hruby & Balse (2000) Curr Med Chem 7:945-970.
{18} Ribka & Rich (1998) Curr Opin Chem Biol 2:441-452.
{19} Chakraborty et al. (2002) Curr Med Chem 9:421-435.
{20} Computer-Assisted Lead Finding and Optimization (eds. Testra & Folkers, 1997)
{21} Available from Molecular Simulations Inc (http://www.msi.com/):
{22} Davic & Lawrence (1992) Proteins 12:31-41.
{23} Caflish et al. (1993) J Med. Chem. 36:2142-67
{24} Eisen et al. (1994) Proteins: Str. Funct. Genet. 19:199-221.
{25} Böhm (1992) J. Comp. Aided Molec. Design 6:61-78.
{26} Gehlhaar et al. (1995) J. Med. Chem. 38:466-72.
{27} Moon & Howe (1991) Proteins: Sir. Funct. Genet. 11:314-328.
{28} Available from http://chem.leeds.ac.uk/ICAMS/SPROUT.html.
{29} Lauri & Bartlett (1994) Comp. Aided Mol. Design 8:51-66.
{30} Available from Tripos Inc (http://www.tripos.com).
{31} Rotstein et al. (1993) J. Med Chem. 36:1700.
{32} Lai (1996) J. Chem. Inf. Comput. Sci. 36:1187-1194.
{33} Gennaro (2000) Remington: The Science and Practice of pharmacy. 20th ed., ISBN: 0683306472
{34} Almeida & Alpar (1996) J. Drug Targeting 3:455-467.
{35} WO99/27961.
{36} WO02/074244.
{37} WO02/064162.
{38} WO03/028760.
{39} Current Protocols in Molecular Biology (F. M. Ausubel et al., eds., 1987) Supplement 30.
{40} Smith & Waterman (1981) Adv. Appl. Math. 2: 482-489.
{41} WO 03/010194.
{42} Bosch et al. (2003) J Virol 77:8801-11.

1. A compound that can bind to the heptad repeat sequence(s) HR1 and/or HR2 of the NadA adhesin on the surface of a meningococcus, thereby inhibiting the ability of the meningococcus either to infect a host organism or to spread an existing infection.

2. The compound of claim 1, wherein, with reference to the numbering of SEQ ID NO:1, the HR1 sequence is residues 117-152 and the HR2 sequence is residues 261-299.

3. A compound that can bind to the heptad repeat sequence(s) HR1 and/or HR2 of the NadA adhesin on the surface of a haemophilus bacterium, thereby inhibiting the ability of the haemophilus either to infect a host organism or to spread an existing infection.

4. The compound of claim 3, wherein, with reference to the numbering of SEQ ID NO: 35, the HR1 sequence is residues 71-91 and the HR2 sequence is residues 120-183.

5. The compound of claim 1, wherein the compound is an oligopeptide.

6. The oligopeptide of claim 5, consisting of no more than 50 amino acids.

7. The oligopeptide of claim 5 or claim 6, comprising a fragment of 5 or more consecutive amino acids from an amino acid sequence selected from the group consisting of: SEQ ID NO: 12, SEQ ID NO:13, SEQ ID NO:14, SEQ ID NO:15, SEQ ID NO:16, SEQ ID NO:17, SEQ ID NO: 39 and SEQ ID NO: 40.

8. The oligopeptide of claim 7, provided that said fragment includes m amino acid substitutions when compared to said SEQ ID, where m is 1 or more.

9. The oligopeptide of any one of claims 4 to 6, comprising one or more of amino acid sequences SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 29, SEQ ID NO: 30, SEQ ID NO: 31, SEQ ID NO: 37 and SEQ ID NO: 38.

10. A polypeptide of formula NH2-A-(B-C)n-D-COOH, wherein: n is an integer between 1 and 5, -A is an optional N-terminus sequence consisting of 1 or more amino acids, (each) -B- is an amino acid sequence comprising a fragment of 5 or more consecutive amino acids from SEQ ID NO:12, SEQ ID NO:13, SEQ ID NO: 14, SEQ ID NO: 15, SEQ ID NO: 16 and/or SEQ ID NO:17; (each) -C- is an optional linker sequence consisting of 1 or more amino acids; and -D- is an optional C-terminus sequence consisting of 1 or more amino acids.

11. A peptidomimetic compound of the oligopeptide of claim 3, wherein the peptidomimetic compound has anti-meningococcal and/or anti-haemophilus activity.

12. A pharmaceutical composition comprising (a) a compound of claim 1 and (b) a pharmaceutical carrier.

13. A method for treating a patient suffering from a meningococcal or haemophilus infection, comprising administering to the patient the pharmaceutical composition of claim 12.

14. (canceled)

15. (canceled)

16. A mutant NadA protein, wherein the mutant protein lacks one or more of the HR1, HR2 or fusion sequences.

17. A mutant NadA protein of claim 16, wherein the protein does not contain one or more of the following amino acid sequences: (i) a sequence which has at least 50% identity to SEQ ID NO: 3; (ii) a sequence which has at least 50% identity to SEQ ID NO: 5; (iii) a sequence which has at least 50% identity to SEQ ID NO: 7; (iv) a sequence which has at least 50% identity to SEQ ID NO: 10.

18. The mutant of claim 17, wherein amino acid sequences (i), (ii), (iii) and (iv) are each at least 10 amino acids long.

19. The mutant NadA protein of claim 16, comprising amino acid sequence-A-B-C-D-E-F-G-H-I-, wherein: -A- is an amino acid sequence with at least 50% sequence identity to amino acids 26-116 of SEQ ID NO: 1; -B- is an amino acid sequence with at least 50% sequence identity to amino acids 117-152 of SEQ ID NO: 1; -C- is an amino acid sequence with at least 50% sequence identity to amino acids 153-180 of SEQ ID NO: 1; -D- is an amino acid sequence with at least 50% sequence identity to amino acids 181-199 of SEQ ID NO: 1; -E- is an amino acid sequence with at least 50% sequence identity to amino acids 200-260 of SEQ ID NO: 1; -F- is an amino acid sequence with at least 50% sequence identity to amino acids 261-275 of SEQ ID NO: 1; -G- is an amino acid sequence with at least 50% sequence identity to amino acids 276-277 of SEQ ID NO: 1; -H- is an amino acid sequence with at least 50% sequence identity to amino acids 278-299 of SEQ ID NO: 1; -I- is an amino acid sequence with at least 50% sequence identity to amino acids 300-364 of SEQ ID NO: 1, provided that at least one of -B-, -D-, -F- or -H- is not present in said protein.

20. A mutant NadA protein, wherein the mutant protein lacks one or more of the HR1, HR2 or fusion sequences.

21. Nucleic acid encoding the mutant protein of any one of claims 16 to 20.

22. A bacterium which expresses the nucleic acid of claim 21.


SEQUENCE LISTING (40 sequences)

SEQ ID NO: 1 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 2 - protein, 405 aa, Neisseria meningitidis
MKHFPSKVLTTAILATFCSGALAATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVGRFNVTAAVGGYKSESAVAIGTGFRFTENFAAKAGVAVGTSSGSSAAYHVGVNYEW

SEQ ID NO: 3 - protein, 19 aa, Neisseria meningitidis
AEAFNDIADSLDETNTKAD

SEQ ID NO: 4 - protein, 36 aa, Neisseria meningitidis
  VARIANT 24: Xaa = Glu or Ala
IEKLTTKLADTDAALADTDAALDXTTNALNKLGENI

SEQ ID NO: 5 - protein, 36 aa, Neisseria meningitidis
IEKLTTKLADTDAALADTDAALDETTNALNKLGENI

SEQ ID NO: 6 - protein, 36 aa, Neisseria meningitidis
IEKLTTKLADTDAALADTDAALDATTNALNKLGENI

SEQ ID NO: 7 - protein, 15 aa, Neisseria meningitidis
IATNKADIAKNSARI

SEQ ID NO: 8 - protein, 17 aa, Neisseria meningitidis
IATNKDNIAKKANSADV

SEQ ID NO: 9 - protein, 22 aa, Neisseria meningitidis
  VARIANT 4: Xaa = Thr or Asn
LDKXVXXLRKETRQGLAEQAAL

SEQ ID NO: 10 - protein, 22 aa, Neisseria meningitidis
LDKNVANLRKETRQGLAEQAAL

SEQ ID NO: 11 - protein, 22 aa, Neisseria meningitidis
LDKTVSDLRKETRQGLAEQAAL

SEQ ID NO: 12 - protein, 46 aa, Neisseria meningitidis
AAESEIEKLTTKLADTDAALADTDAALDETTNALNKLGENITTFAE

SEQ ID NO: 13 - protein, 46 aa, Neisseria meningitidis
AAESEIEKLTTKLADTDAALADTDAALDATTNALNKLGENITTFAE

SEQ ID NO: 14 - protein, 25 aa, Neisseria meningitidis
DIKADIATNKADIAKNSARIDSLDK

SEQ ID NO: 15 - protein, 27 aa, Neisseria meningitidis
DIKADIATNKDNIAKKANSADVYTREE

SEQ ID NO: 16 - protein, 32 aa, Neisseria meningitidis
ARIDSLDKNVANLRKETRQGLAEQAALSGLFQ

SEQ ID NO: 17 - protein, 32 aa, Neisseria meningitidis
TRLNGLDKTVSDLRKETRQGLAEQAALSGLFQ

SEQ ID NO: 18 - protein, 1255 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDFGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 19 - protein, 19 aa, mouse hepatitis virus
KMIASAFNNALGAIQDGFD

SEQ ID NO: 20 - protein, 19 aa, bovine coronavirus
KLIANAFNNALDAIQEGFD

SEQ ID NO: 21 - protein, 19 aa, Feline Infectious Peritonitis Virus
KILANAFNNAIGNITLALG

SEQ ID NO: 22 - protein, 19 aa, transmissible gastroenteritis coronavirus
QILASAFNQAIGNITQSFG

SEQ ID NO: 23 - protein, 19 aa, Avian infectious bronchitis virus
EKIAASFNKAIGHMQEGFR

SEQ ID NO: 24 - protein, 19 aa, Human coronavirus 229E
KILAASFNKAMTNIVDAFT

SEQ ID NO: 25 - protein, 19 aa, Human coronavirus OC43
KLIANAFNNALYAIQEGFD

SEQ ID NO: 26 - protein, 19 aa, SARS coronavirus
KQIANQFNKAISQIQESLT

SEQ ID NO: 27 - protein, 18 aa, Artificial Sequence
  Coronavirus consensus sequence
KIIANAFNNAIGNIQEGF

SEQ ID NO: 28 - protein, 18 aa, Neisseria meningitidis
KHAEAFNDIADSLDETNT

SEQ ID NO: 29 - protein, 42 aa, Neisseria meningitidis
ESEIEKLTTKLADTDAALADTDAALDATTNALNKLGENITTF

SEQ ID NO: 30 - protein, 23 aa, Neisseria meningitidis
KADIATNKDNIAKKANSADVYTR

SEQ ID NO: 31 - protein, 28 aa, Neisseria meningitidis
LNGLDKTVSDLRKETRQGLAEQAALSGL

SEQ ID NO: 32 - protein, 24 aa, Neisseria meningitidis
TVDKHAEAFNDIADSLDETNTKAD

SEQ ID NO: 33 - protein, 21 aa, Neisseria meningitidis
KHAEAFNDIADSLDETNTKAD

SEQ ID NO: 34 - protein, 19 aa, SARS coronavirus
KIIANAFNNAIGNIQEGLT

SEQ ID NO: 35 - protein, 256 aa, Haemophilus influenzae biogroup aegyptius
MKRNLLKQSVIAVLIGGTTVSNYALAQAQAQAQVKKDELSELKKQVKEMDAAIDGILDDNIAYEAEVDAKLDQHSAALGRHTNRLNNLKTIAEKAKGDSSEALDKIEALEEQNDEFLADITALEEGVDGLDDDITGIQDNISDIEDDINQNSADIATNTAAIATHTQRLDNLDNRVNNLNKDLKRGLAAQAALNGLFQPYNVGKLNLTAAVGGYKSQTAVAVGTGYRYNENIAAKAGVAFTHGGSATYNVGVNFEW

SEQ ID NO: 36 - protein, 17 aa, Haemophilus influenzae biogroup aegyptius
AAIDGILDDNIAYEAEV

SEQ ID NO: 37 - protein, 21 aa, Haemophilus influenzae biogroup aegyptius
LDQHSAALGRHTNRLNNLKTI

SEQ ID NO: 38 - protein, 64 aa, Haemophilus influenzae biogroup aegyptius
ITALEEGVDGLDDDITGIQDNISDIEDDINQNSADIATNTAAIATHTQRLDNLDNRVNNLNKDL

SEQ ID NO: 39 - protein, 40 aa, Haemophilus influenzae biogroup aegyptius
VDAKLDQHSAALGRHTNRLNNLKTIAEKAKGDSSEALDKI

SEQ ID NO: 40 - protein, 69 aa, Haemophilus influenzae biogroup aegyptius
FLADITALEEGVDGLDDDITGIQDNISDIEDDINQNSADIATNTAAIATHTQRLDNLDNRVNNLNKDLK